(19) United States
(12) Patent Application Publication
(10) Pub. No.: US 2022/0152051 A1
(43) Pub. Date: May 19, 2022

(54) APPLICATION OF GLYCOCHOLIC ACID IN PREPARATION OF ANTITUMOR DRUGS

(71) Applicant: DALIAN UNIVERSITY, Dalian (CN)

(72) Inventors: Wenbo LIANG, Dalian (CN); Rongrong XIANG, Dalian (CN); Yiming JIANG, Dalian (CN); Yuchun TONG, Dalian (CN); Jie SONG, Dalian (CN)

(21) Appl. No.: 17/438,500

(22) PCT Filed: Mar. 15, 2019
(86) PCT No.: PCT/CN2019/078305

§ 371 Date: Dec. 7, 2021

(51) Int. Cl. A61K 31/575; A61P 35/00

(52) U.S. Cl. CPC A61K 31/575; A61P 35/00

(57) ABSTRACT

The present invention relates to a use of a chemical drug in the field of medicine, and relates in particular to an application of glycocholic acid in the preparation of anti-tumor drugs. Provided by the present invention is a new application field for glycocholic acid, wherein glycocholic acid can be used in the preparation of anti-tumor drugs; the main applicable tumors comprise breast cancer, ovarian cancer, endometrial cancer, or a combination thereof; and glycocholic acid is used as an effective active ingredient in anti-tumor drugs. The present invention is beneficial in developing new anti-tumor drugs.

### TECHNICAL FIELD

The present invention relates to a use of a chemical drug in the field of medicine, and relates in particular to application of glycocholic acid in the preparation of anti-tumor drugs.

### BACKGROUND

The malignant tumor is one of the major diseases that seriously endanger human health, a total of 8.4 million people died of the malignant tumor worldwide in 2010 according to the report of the World Health Organization (WHO), and it is predicted that this number may increase to 10 million by 2020. About 1.8 million new cases of the malignant tumor occur each year in China, about 1.3 million people died of the malignant tumor, and the malignant tumor has become the leading cause of death in China.

The medical treatment for tumors has a short history in modern medical oncology, so far less than 70 years, but the medical treatment of which the history is less than one century has made many significant results, making the medical treatment an indispensable part in the comprehensive tumor treatment. At present, most of the anti-tumor drugs used clinically kill tumor cells and normal cells of the body at the same time, which is most obvious in the immune system and hematopoietic system. Eventually lead to long-term drug intolerance, tumor recurrence and metastasis, and tumor drug resistance. Therefore, in recent years, people have been committed to developing new anti-tumor drugs, most of which have clear targets and have little or no harm to the immune and hematopoietic system.

Glycocholic acid is conjugated cholic acid, and is generated by conjugating cholic acid with glycine, and the cholic acid is a metabolic product of cholesterol in livers.

The physiological effect of glycocholic acid is mainly to promote digestion and absorption of fat in intestinal tracts, but the application of glycocholic acid as a drug, especially in antitumor drugs, has not been reported.

### SUMMARY

Aiming at the defects in the prior art, in order to sufficiently utilize the active components of glycocholic acid, the present invention aims to provide application of glycocholic acid in the preparation of an anti-tumor drug.

In order to achieve the above-mentioned object of the invention, the present invention provides the application of glycocholic acid in the anti-tumor drug, wherein a molecular structural formula of glycocholic acid is as follows:

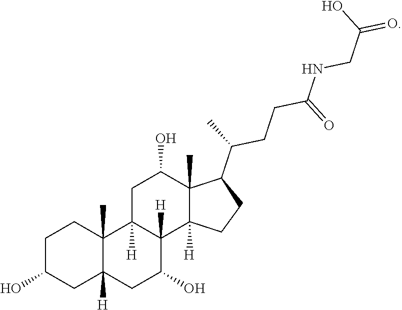

In the application of glycocholic acid in the preparation of the anti-tumor drug, the anti-tumor drug is an anti-tumor for human beings.

In the application of glycocholic acid in the preparation of the anti-tumor drug, the anti-tumor drug is an anti-tumor drug for animals.

In the application of glycocholic acid in the preparation of the anti-tumor drug, animals to which the anti-tumor drug is applied are primates and rodentia animals.

In the application of glycocholic acid in the preparation of the anti-tumor drug, the tumors involved are malignant tumors such as breast cancer, ovarian cancer, endometrial cancer and the like.

In the application of glycocholic acid in the preparation of the anti-tumor drug, the application of the anti-tumor drug includes application in a tumor cell growth inhibitor.

The present invention further provides an anti-tumor drug containing glycocholic acid and a derivative thereof.

The above-mentioned anti-tumor drug containing glycocholic acid and the derivative thereof, the anti-tumor drug includes an effective therapeutic dose of glycocholic acid and the remainder of a pharmaceutically acceptable carrier.

The above-mentioned anti-tumor drug containing glycocholic acid and the derivative thereof, the effective therapeutic dose is 0.4 to 1.6 nmol/L for 4T1 (mouse breast cancer cells), 0.16 to 1.28 μmol/L for MCF-7 (human breast cancer cells), and 0.08 to 1.32 μmol/L for MDA-MB-468 (human breast cancer cells).

The above-mentioned anti-tumor drug containing glycocholic acid and the derivative thereof are an oral preparation, a suppository, an injection or a transdermal agent. The present invention provides a new application field for glycocholic acid; glycocholic acid can be used in the preparation of the anti-tumor drugs; the main applicable tumors include breast cancer (for example, 4T1, MCF-7, and MDA-MB-468), ovarian cancer (for example, NIH:OVCAR-3), endometrial cancer (for example, RL95-2), or a combination thereof and glycocholic acid is used as an effective active ingredient in the anti-tumor drugs, and the effective therapeutic dose thereof is 0.4 to 1.6 nmol/L for 4T1, 0.16 to 1.28 μmol/L for MCF-7, and 0.08 to 1.32 μmol/L for MDA-MB-468; and the present invention is beneficial in developing new anti-tumor drugs.

### BRIEF DESCRIPTION OF THE DRAWINGS

FIG. 1 shows an inhibition ratio in each time period of a 4T1 mouse breast cancer cell group in which 0.8 nmol/L of glycocholic acid is added (compared with a negative control group, *P<0.05).

FIG. 2 shows an inhibition ratio of 4T1 mouse breast cancer cell in each group at 12 hours (compared with a negative control group, *P<0.05).

FIG. 3 shows an inhibition ratio in each time period of an MCF-7 human breast cancer cell group in which 1.28 μmol/L is added (compared with a negative control group, *P<0.05).

FIG. 4 shows an inhibition ratio of MCF-7 human breast cancer cell in each group at 36 hours (compared with a negative control group, *P<0.05).

FIG. 5 shows an inhibition ratio in each time period of an MDA-MB-468 human breast cancer cell group in which 1.32 μmol/L is added (compared with a negative control group, *P<0.05).

FIG. 6 shows an inhibition ratio of MDA-MB-468 human breast cancer cell in each group at 36 hours (compared with a negative control group, *P<0.05).

FIG. 7 shows an inhibition ratio in each time period of a NIH:OVCAR-3 human ovarian cancer cell group in which 1.28 μmol/L is added (compared with a negative control group, *P<0.05).

FIG. 8 shows an inhibition ratio of NIH:OVCAR-3 human ovarian cancer cell in each group at 48 hours (compared with a negative control group, *P<0.05).

FIG. 9 shows an inhibition ratio in each time period of an RL95-2 human endometrial cancer cell group in which 1.28 μmol/L is added (compared with a negative control group, *P<0.05).

FIG. 10 shows an inhibition ratio of RL95-2 human endometrial cancer cell in each group at 36 hours (compared with a negative control group, *P<0.05).

FIG. 11 shows an inhibition ratio in each time period of a 4T1 mouse breast cancer cell group in which 0.7 nmol/L of glycocholic acid is added (compared with a negative control group, *P<0.05).

FIG. 12 shows an inhibition ratio of 4T1 mouse breast cancer cell in each group at 12 hours (compared with a negative control group, *P<0.05).

FIG. 13 shows an FXR gene expression curve at 12 h of the 4T1 mouse breast cancer cell group in which 0.8 nmol/L is added.

FIG. 14 shows the FXR gene expression histogram of the 4T1 mouse breast cancer cell group in which 0.8 nmol/L is added at 12 h (compared with a negative control group, *P<0.05).

FIG. 15 shows an FXR protein expression blotting stripe at 12 h of the 4T1 mouse breast cancer cell group in which 0.8 nmol/L is added.

FIG. 16 shows the FXR protein expression histogram at 12 h of the 4T1 mouse breast cancer cell group in which 0.8 nmol/L is added (compared with a negative control group, *P<0.05).

### DETAILED DESCRIPTION

The present disclosure will be further illustrated below in combination with specific embodiments.

Glycocholic acid used in the embodiments is purchased from an Aladdin reagent, and product number G131002.

### Example 1 In-Vitro Anti-Tumor Experiment of Glycocholic Acid

1. Cell line: 4T1 (mouse breast cancer cells), MCF-7 (human breast cancer cells), MDA-MB-468 (human breast cancer cells), NIH:OVCAR-3 (human ovarian cancer cells), and RL95-2 (human endometrial cancer cells) (all the above are purchased from the Shanghai Cell Bank of the Chinese Academy of Sciences).

2. Culture medium: RPMI-1640 (Gibco, the article number: 31800022); and a high-glucose DMEM culture medium (Gibco, the article number: 11965092).

3. Method

3.1 Cells at a logarithmic growth phase are collected, the concentration of the cells is respectively regulated into 5×10⁴ cells/ml by the 1640 culture medium and the DMEM culture medium, and 100 μl of cell suspension is added into a 96-well plate.

3.2 After culture is carried out for 24 h at the temperature of 37° C. at 5% CO2, the concentration gradient of glycocholic acid respectively of 0.1 nmol/L, 0.2 nmol/L, 0.4 nmol/L, 0.8 nmol/L, 1.6 nmol/L, 3.2 nmol/L and 6.4 nmol/L is added into 4T1 mouse breast cancer cells, the concentration gradient of glycocholic acid respectively of 0.02 μmol/L, 0.04 μmol/L, 0.08 μmol/L, 0.16 μmol/L, 0.32 μmol/L, 0.64 μmol/L and 1.28 μmol/L is added into MCF-7 cells, the concentration gradient of glycocholic acid respectively of 0.02 μmol/L, 0.04 μmol/L, 0.08 μmol/L, 0.16 μmol/L, 0.33 μmol/L, 0.66 μmol/L and 1.32 μmol/L is added into MDA-MB-468 cells, the concentration gradient of glycocholic acid respectively of 0.02 μmol/L, 0.04 μmol/L, 0.08 μmol/L, 0.16 μmol/L, 0.32 μmol/L, 0.64 μmol/L and 1.28 μmol/L is added into NIH:OVCAR-3 cells, the concentration gradient of glycocholic acid respectively of 0.02 μmol/L, 0.04 μmol/L, 0.08 μmol/L, 0.16 μmol/L, 0.32 μmol/L, 0.64 μmol/L and 1.28 μmol/L is added into RL-95-2 cells, set up five duplicated wells and meanwhile, set up a blank group and a control group.

3.3 Add CCK-8 reagent after 12 h, 24 h, 36 h, 48 h and 60 h, and incubate for 3 h, and after incubation is completed, the absorption value of each well is measured at OD450 nm of the microplate reader.

3.4 An inhibition rate of each concentration of glycocholic acid for tumor cells is obtained by utilizing a formula of

$$inhibition rate = \frac{( control well OD - dosing well OD )}{( control well OD - blank well OD )} \times 100 \% .$$

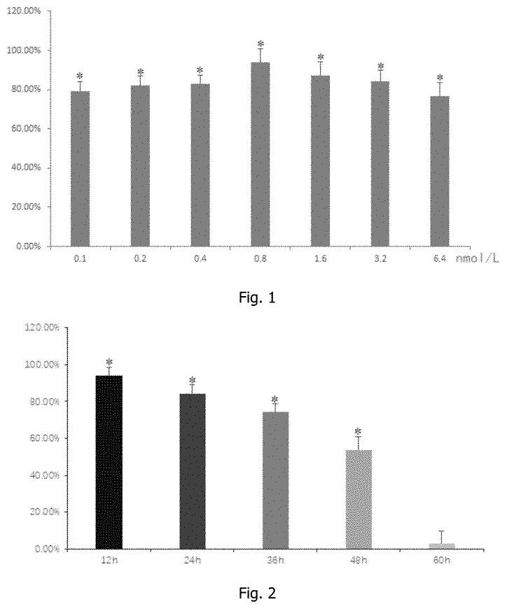

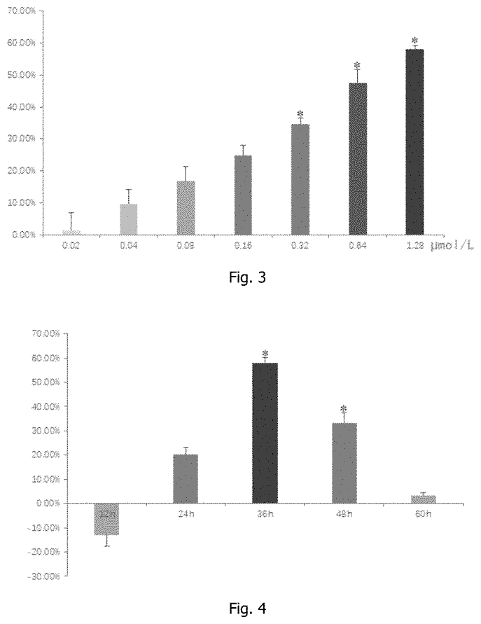

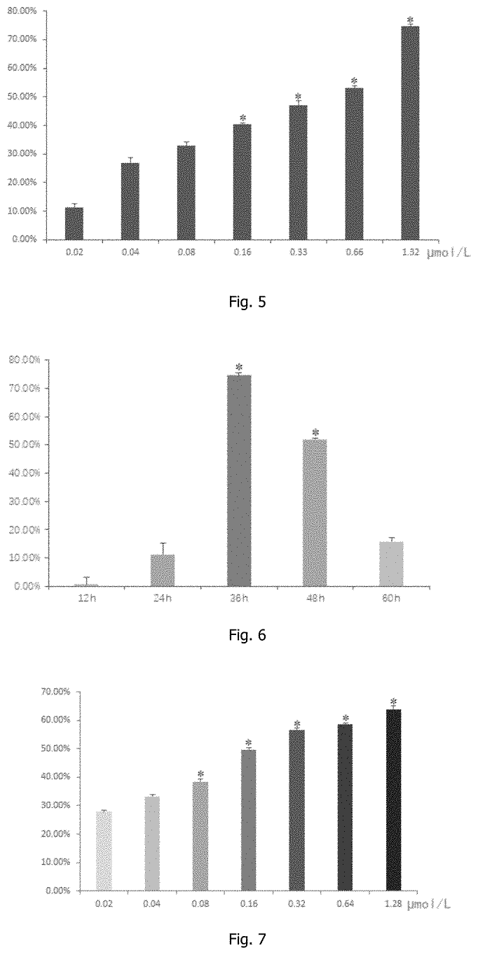

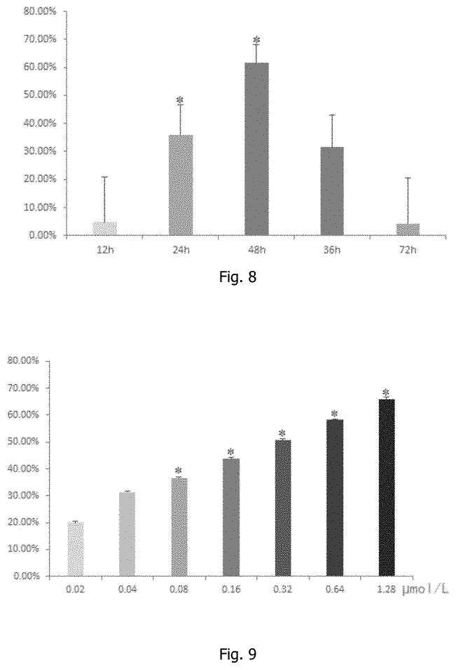

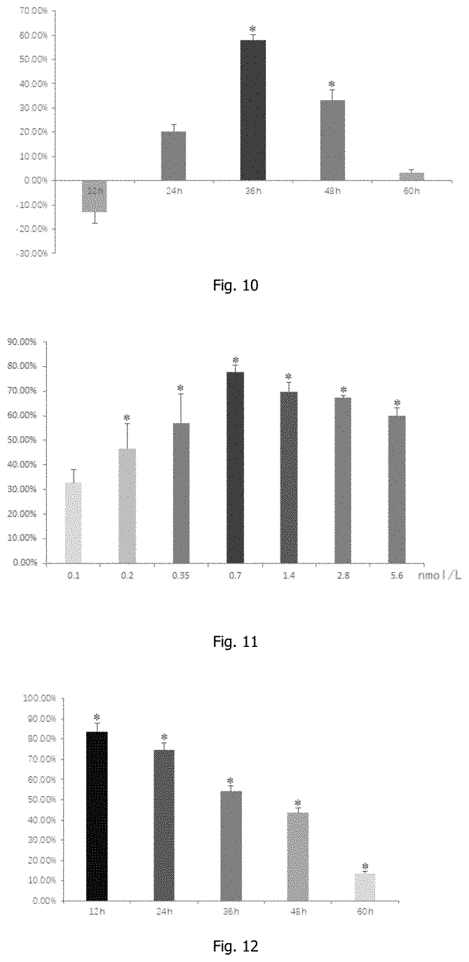

4. Result: when the 4T1 mouse breast cancer cells reach the highest point of the inhibition rate at 12 h, the concentration of glycocholic acid is 0.8 nmol/L, as shown in FIG. 1 and FIG. 2, its IC50 is between 0.01 nmol/L and 0.1 nmol/L; when the MCF-7 cells reach the highest point of the inhibition rate at 36 h, the concentration of glycocholic acid is 1.28 μmol/L, as shown in FIG. 3 and FIG. 4, its IC50 is respectively between 0.64 μmol/L and 1.00 μmol/L; when the MDA-MB-468 cells reach the highest point of the inhibition rate at 36 h, the concentration of glycocholic acid is 1.32 μmol/L, as shown in FIG. 5 and FIG. 6, its IC50 is respectively between 0.33 μmol/L and 0.66 μmol/L; when the NIH-OVCAR-3 cells reach the highest point of the inhibition rate at 48 h, the concentration of glycocholic acid is 1.28 μmol/L, as shown in FIG. 7 and FIG. 8, its IC50 is respectively between 0.16 μmol/L and 0.32 μmol/L; and when the RL95-2 cells reach the highest point of the inhibition rate at 36 h, the concentration of glycocholic acid is 1.28 μmol/L, as shown in FIG. 9 and FIG. 10, its IC50 is respectively between 0.32 μmol/L and 0.64 μmol/L.

### Example 2 In-Vitro Anti-Tumor Experiment of Sodium Glycocholate

1. Cell line: 4T1 (mouse breast cancer cells) (all the above are purchased from the Shanghai Cell Bank of the Chinese Academy of Sciences).

2. Culture Medium: RPMI-1640 (Gibco, the article number: 31800022).

3. Method

3.1 Cells at a logarithmic growth phase are collected, the concentration of the cells is regulated into 5×10⁴ cells/ml by the 1640 culture medium, and 100 μl of cell suspension is added into a 96-well plate.

3.2 After culture is carried out for 24 h at the temperature of 37° C. at 5% CO2, the concentration gradient of sodium glycocholate respectively of 0.1 nmol/L, 0.25 nmol/L, 0.35 nmol/L, 0.7 nmol/L, 1.4 nmol/L, 2.8 nmol/L and 5.6 nmol/L is added into 4T1 mouse breast cancer cells, set up five duplicated wells, and meanwhile, set up a blank group and a control group.

3.3 Add CCK-8 reagent after 12 h, 24 h, 36 h, 48 h and 60 h, incubate for 3 h, and after incubation is completed, the absorption value of each well is measured at OD450 nm of the microplate reader.

3.4 An inhibition rate of each concentration of sodium glycocholate for tumor cells is obtained by utilizing a formula of

$$tumor inhibition rate = \frac{\begin{matrix}average tumor mass in \\ the control group - \\ average tumor mass in \\ the experimenting group\end{matrix}}{\begin{matrix}average tumor mass in \\ the control group\end{matrix}} \times 100 \% .$$

4. Result: when the 4T1 mouse breast cancer cells reach the highest point of the inhibition rate at 12 h, the concentration of sodium glycocholate is 0.7 nmol/L, as shown in FIG. 11 and FIG. 12, its IC50 is between 0.01 nmol/L and 0.1 nmol/L.

### Example 3 Measurement of Tumor Inhibition Rate of Glycocholic Acid by Animal Model

1. Materials: 30 female 10-week-old BABL/c mice purchased from the SPF Experimental Animal Center of the Dalian Medical University; and 4T1 tumor cells purchased from the Shanghai Cell Bank of the Chinese Academy of Sciences.

2. Method:

2.1 Modeling: 4T1 mouse breast cancer cells at a logarithmic growth phase are collected, and the concentration of the cells is adjusted to 2×10⁶ cells/ml with PBS; and 0.1 ml of cell suspension is injected under the right armpits of the mice by a sterile syringe.

2.2 Administration: intragastric administration of 15 mg/kg of glycocholic acid is started on the second day after modeling (twice/day), 0.2 mL each time, the equal volume of distilled water is administrated to the control group, and administration is continuously carried out for two weeks. The mice are fasted on the night of the last administration, the mice are sacrificed on the fifteenth day by a cervical dislocation method, tumor tissues under the armpits of the mice are aseptically stripped and weighed, and the tumor inhibition rate is calculated according to a formula of

$$inhibition rate = \frac{( control well OD - dosing well OD )}{( control well OD - blank well OD )} \times 100 \% .$$

3. Result: the average mouse tumor mass in the experiment group is 0.247±0.08 g, the average mouse tumor mass in the control group is 0.431±0.167 g, the tumors of the mice in the experiment group are obviously smaller than those of the control group, and the tumor inhibition rate reaches 42.83%.

### Example 4 Real-Time PCR Determination of Glycocholic Acid Specific Target RNA Expression in Tumor Cells

1. Materials: 4T1 mouse breast cancer cells purchased from the Shanghai Cell Bank of the Chinese Academy of Sciences.

2. Method: RNA of the 4T1 cells intervened by glycocholic acid is extracted, and after reverse transcription, the Real-time RCP is carried out to measure the changes in the RNA expression of the specific target of glycocholic acid.

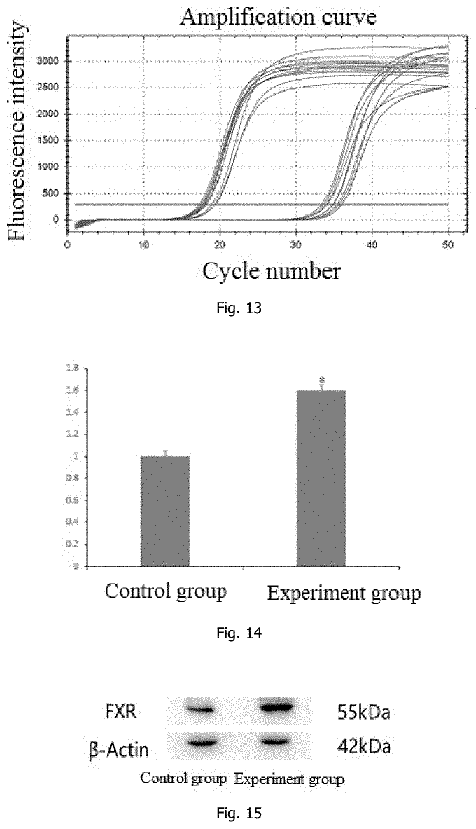

3. Result: after intervention of glycocholic acid, the expression of related RNA is improved by 1.5 times, as shown in FIG. 13 and FIG. 14. The RNA level confirmed the results of the intervention of glycocholic acid on tumor cells, and revealed the mechanism of glycocholic acid inhibiting the growth of tumor cells.

### Example 5 Western Blot Experiment to Determine the Changes of Expression of the Specific Target Protein of Glycocholic Acid in Tumor Cells after Intervention of Glycocholic Acid

1. Materials: 4T1 mouse breast cancer cells purchased from the Shanghai Cell Bank of the Chinese Academy of Sciences.

2. After proteins of 4T1 cells intervened by glycocholic acid are extracted, by degeneration treatment, changes in the expression of the related proteins are measured according to the Western blot flow.

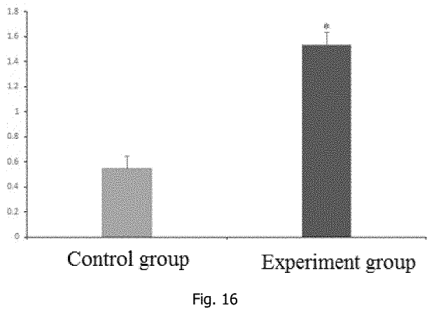

Result: the Western blot experiment shows that the expression of the specific target protein of the glycocholic acid in tumor cells is increased, as shown in FIG. 15 and FIG. 16. The protein level confirmed the effect of glycocholic acid on tumor cells, and revealed the mechanism of glycocholic acid inhibiting the growth of tumor cells.

1. Application of glycocholic acid in a preparation of an anti-tumor drug, wherein the anti-tumor drug is an anti-tumor drug for human beings or the anti-tumor drug for animals, and the tumor is one or more of breast cancer, ovarian cancer and endometrial cancer.

2. The application according to claim 1, wherein the animals are primates and rodentia animals.

3. The application according to claim 1, wherein the anti-tumor drug is a tumor cell growth inhibitor.

4. An anti-tumor drug containing glycocholic acid and a derivative thereof converted into glycocholic acid in vivo, wherein the anti-tumor drug comprises an effective therapeutic dose of glycocholic acid and a remainder of a pharmaceutically acceptable carrier.

5. The anti-tumor drug containing glycocholic acid and the derivative thereof according to claim 4, wherein an effective therapeutic dose for a mouse breast cancer cell 4T1 is 0.4 to 1.6 nmol/L.

6. The anti-tumor drug containing glycocholic acid and the derivative thereof according to claim 4, wherein an effective therapeutic dose for a human breast cancer cell MCF-7 is 0.16 to 1.28 μmol/L.

7. The anti-tumor drug containing glycocholic acid and the derivative thereof according to claim 4, wherein an effective therapeutic dose for a human breast cancer cell MDA-MB-468 is 0.08 to 1.32 μmol/L.

8. The anti-tumor drug containing glycocholic acid and the derivative thereof according to claim 4, wherein the drug is an oral preparation, a suppository, an injection or a transdermal agent.
